(19) United States
(12) Patent Application Publication
(10) Pub. No.: US 2026/0284364 A1
(43) Pub. Date: Sep. 24, 2026

(54) REMOVABLE ACCESS SHEATH

(71) Applicant: BOSTON SCIENTIFIC SCIMED, INC., Maple Grove, MN (US)

(72) Inventors: Nicholas Lee Tassoni, Andover, MN (US); Daniel Frank Massimini, Brooklyn Park, MN (US); Jarrod Kenneth Neuharth, Coon Rapids, MN (US)

(73) Assignee: BOSTON SCIENTIFIC SCIMED, INC., Maple Grove, MN (US)

(21) Appl. No.: 19/566,237

(22) Filed: Mar. 13, 2026

Related U.S. Application Data
(60) Provisional application No. 63/775,514, filed on Mar. 21, 2025.

(51) Int. Cl. A61M 25/06; A61M 25/00; A61M 60/865

(52) U.S. Cl. CPC A61M 25/0668; A61M 25/0097; A61M 2025/0675; A61M 60/865

(57) ABSTRACT

Example access sheaths are disclosed. An example access sheath for a medical device includes an elongate shaft having a distal end region, a proximal end region and a wall. Further, the access sheath includes a hub coupled to the proximal end region of the elongate shaft and a first pull cord coupled to the hub, wherein the first pull cord is configured to translate along the elongate shaft from the proximal end region to the distal end region. Further, translation of the first pull cord along the elongate shaft cuts the wall of the elongate shaft from the proximal end region to the distal end region.

### CROSS-REFERENCE TO RELATED APPLICATION

This application claims the benefit of U.S. patent application Ser. No. 63/775,514, filed Mar. 21, 2025, entitled “REMOVABLE ACCESS SHEATH”, which is incorporated by reference herein in its entirety.

### TECHNICAL FIELD

The disclosure relates generally to medical devices and more particularly to medical devices that are adapted for use in percutaneous medical procedures.

### BACKGROUND

In some instances, performing percutaneous medical procedures may require the insertion and/or maneuvering of relatively large medical devices through a patient's vasculature. Therefore, in some instances a relatively large vascular access sheath may be utilized to facilitate the insertion of relatively large medical devices into a patient's vasculature. However, inserting a relatively large medical device into the vasculature through a large bore access sheath for an extended time period may, for example, lead to tissue damage (e.g., leg ischemia) due to a reduction in blood flow. Tissue damage resulting from placement of a relatively large medical device into the vasculature for an extended time period may be lessened by minimizing the size of the access sheath used to access the vessel. It may be desirable to configure a relatively large bore access sheath capable of removal for replacement with a smaller access sheath.

### SUMMARY

This disclosure provides design, material, manufacturing method, and use alternatives for medical devices. An example access sheath for a medical device includes an elongate shaft having a distal end region, a proximal end region and a wall. Further, the access sheath includes a hub coupled to the proximal end region of the elongate shaft and a first pull cord coupled to the hub, wherein the first pull cord is configured to translate along the elongate shaft from the proximal end region to the distal end region. Further, translation of the first pull cord along the elongate shaft cuts the wall of the elongate shaft from the proximal end region to the distal end region.

Alternatively or additionally to any of the examples above, wherein the hub is configured to separate into a first hub member and a second hub member.

Alternatively or additionally to any of the examples above, wherein the first pull cord includes a first end attached to the first hub member.

Alternatively or additionally to any of the examples above, wherein at least a portion of the first pull cord is positioned within the wall of the elongate shaft.

Alternatively or additionally to any of the examples above, further comprising a second pull cord coupled to the hub.

Alternatively or additionally to any of the examples above, wherein the second pull cord is circumferentially offset from the first pull cord.

Alternatively or additionally to any of the examples above, wherein at least a portion of the second pull cord is positioned within the wall of the elongate shaft.

Alternatively or additionally to any of the examples above, wherein the second pull cord is configured to translate along the elongate shaft from the proximal end region to the distal end region, and wherein translation of the second pull cord along the elongate shaft cuts the wall of the elongate shaft from the proximal end region to the distal end region.

Alternatively or additionally to any of the examples above, further comprising a first pull tab releasably coupled to the hub.

Alternatively or additionally to any of the examples above, wherein a first end of the first pull cord is attached to the first pull tab.

Alternatively or additionally to any of the examples above, further comprising a second pull tab releasably coupled to the hub and a second pull cord attached to the second pull tab.

Alternatively or additionally to any of the examples above, further comprising a pull knob releasably attached to the hub.

Alternatively or additionally to any of the examples above, wherein a first end of the first pull cord is attached to the pull knob.

Alternatively or additionally to any of the examples above, further comprising a second pull cord attached to the pull knob.

Alternatively or additionally to any of the examples above, wherein the first hub member is coupled to the second hub member via a splitable membrane.

Alternatively or additionally to any of the examples above, wherein at least a portion of the splitable membrane is positioned along an inner surface of the hub.

Another access sheath for a medical device includes an elongate shaft having a distal end region, a proximal end region and a wall. The access sheath further includes a hub coupled to the proximal end region of the elongate shaft, wherein the hub includes a first hub member configured to separate from a second hub member. The access sheath also includes a pull cord coupled to the hub, wherein the pull cord is configured to translate along the elongate shaft from the proximal end region to the distal end region. Further, translation of the pull cord along the elongate shaft cuts the wall of the elongate shaft from the proximal end region to the distal end region.

Alternatively or additionally to any of the examples above, wherein the hub is configured to separate into a first hub member and a second hub member.

Alternatively or additionally to any of the examples above, wherein the pull cord includes a first end attached to the first hub member.

A method for advancing a medical device into a body lumen includes positioning an access sheath adjacent a body lumen, wherein the access sheath includes an elongate shaft having a distal end region, a proximal end region and a wall. The access sheath also includes a hub coupled to the proximal end region of the elongate shaft and a pull cord attached to the hub, the pull cord configured to cut the wall of the elongate shaft. Further, the method also includes advancing the medical device through the access sheath and into the body lumen and translating the pull cord along the elongate shaft from the proximal end region to the distal end region, wherein translation of the pull cord along the elongate shaft cuts the wall of the elongate shaft from the proximal end region to the distal end region. The method also includes removing the access sheath from the medical device via passing at least a portion of the medical device through the cut portion of the elongate shaft.

The above summary of some examples is not intended to describe each disclosed embodiment or every implementation of the present disclosure. The Figures and Detailed Description, which follow, more particularly exemplify these examples.

### BRIEF DESCRIPTION OF DRAWINGS

FIG. 1 is a perspective view of an example medical device;

FIG. 2 is a perspective view of an example access sheath;

FIG. 3 is a perspective view of the distal end region of an access sheath;

FIG. 4 is a cross-sectional view of an example access sheath in a first configuration;

FIG. 5 is a cross-sectional view of an example access sheath in a second configuration;

FIG. 6 illustrates the access sheath of FIG. 2 in a first configuration;

FIG. 7 illustrates the access sheath of FIG. 2 in a second configuration;

FIG. 8 is a perspective view of an example access sheath;

FIG. 9 illustrates the access sheath of FIG. 8 in a first configuration;

FIG. 10 illustrates the access sheath of FIG. 8 in a second configuration;

FIG. 11 is a perspective view of an example access sheath in a first configuration;

FIG. 12 illustrates the access sheath of FIG. 11 in a second configuration;

FIG. 13 is a perspective view of an example access sheath;

FIG. 14 illustrates the access sheath of FIG. 13 in a first configuration;

FIG. 15 illustrates the access sheath of FIG. 13 in a second configuration; and

FIG. 16 is a perspective view of an example access sheath.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some examples”, “other examples”, etc., indicate that the embodiment described may include one or more particular features, structures, and/or characteristics. However, such recitations do not necessarily mean that all examples include the particular features, structures, and/or characteristics. Additionally, when particular features, structures, and/or characteristics are described in connection with one embodiment, it should be understood that such features, structures, and/or characteristics may also be used connection with other examples whether or not explicitly described unless clearly stated to the contrary.

The following detailed description should be read with reference to the drawings in which similar elements in different drawings are numbered the same. The drawings, which are not necessarily to scale, depict illustrative examples and are not intended to limit the scope of the disclosure.

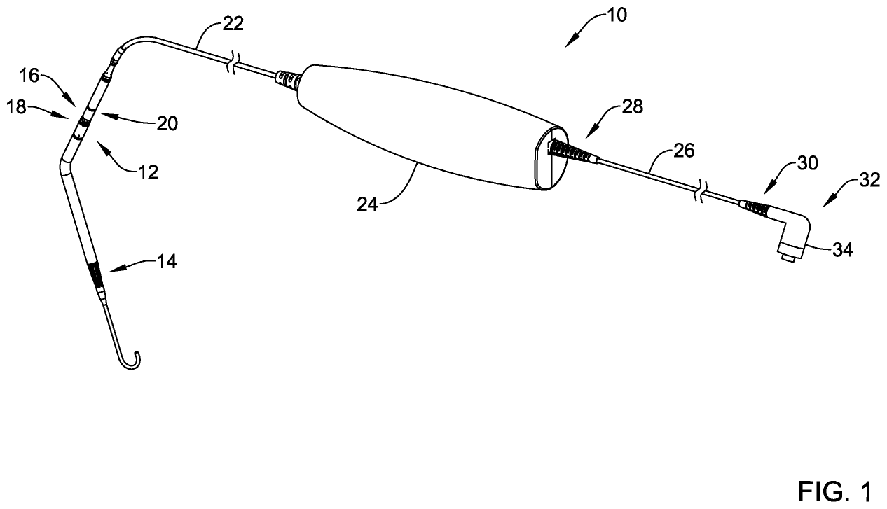

FIG. 1 shows an example percutaneous blood pump 10 (i.e., percutaneous cardiac assist device and/or circulatory support device) for connection to a medical device console (not shown). In the cases of blood pumps such as that depicted in FIG. 1, percutaneous cardiac assist devices and/or circulatory support devices support the pumping action of the heart. These devices may be positioned percutaneously into a patient's heart, such as disposed across a valve opening such as, for example, an aortic valve. Such percutaneous blood pumps 10 assist the heart in pumping blood throughout the cardiovascular system, and the present disclosure provides uses, devices, and methods incorporating such devices as will be described herein. Such percutaneous blood pumps 10 may be connected to a medical device console for operating the blood pump and/or monitoring the operation of the blood pump and/or one or more patient parameters. However, a wide variety of medical devices are utilized to treat patients which are connected to a medical device console for operating the medical device and/or monitoring the operation of the medical device and/or one or more patient parameters. Accordingly, aspects of the present disclosure may be incorporated into a wide variety of medical devices.

Typically, a circulatory support device such as the example blood pump 10 shown in FIG. 1 includes a blood pump housing 12 including a blood inlet 14 at a blood inflow end of the blood pump housing 12 and a blood outlet 16 at a blood outflow end of the blood pump housing 12. The blood pump housing 12 may include an impeller 18 or other blood pumping device configured to convey blood through the blood pump housing 12 from the blood inlet 14 to the blood outlet 16. In some instances, the impeller 18 may be disposed within an impeller housing portion of the blood pump housing 12. The impeller 18 may be constructed of radially oriented blades protruding from a drive shaft and/or drive mechanism, for example. In some instances, the blood inlet 14 may be positioned and/or be positionable in a ventricle of the patient, while the blood outlet 16 may be positioned and/or be positionable in the aorta of the patient, such that the blood pump 10 pumps blood across the aortic valve. The impeller 18 may be driven by a motor 20 or other means of rotating the impeller 18 during operation thereof. In some instances, the motor 20 may be housed within a motor housing portion of the blood pump housing 12. In other instances, the motor 20 may be arranged at another location, if desired. For example, the motor 20 may be located on the blood pump 10 such that it remains outside the body when the blood pump 10 is in use. The motor 20 or other means of rotating the impeller 18 may be configured to rotate the impeller 18 through magnetism, electromagnetism, mechanical force, electrical force, and/or any combination or permutation of the aforementioned, such that the drive shaft and/or drive mechanism rotates the blades of the impeller 18, thereby creating a blood flow to propel blood through the blood pump housing 12 and beyond into the cardiovascular system. The blood pump 10 may include an elongate catheter shaft 22 extending proximally from the blood pump housing 12 for navigating the blood pump 10 to a desired location within the cardiovascular system of the patient. The catheter shaft 22 may extend proximally to a junction box 24, a handle, a manifold, or other structure of the blood pump 10 configured to remain exterior of the patient.

Furthermore, the blood pump 10 may include a cable 26 at a proximal end thereof having a connection interface 32 (e.g., connector) at an end thereof. The cable 26 may have a first, distal end 28 connected to the junction box 24, a handle, a manifold, or other structure of the blood pump 10, and a second, proximal end 30 having the connection interface 32 including a connector 34 that is configured to be connected to a medical device console (e.g., blood pump console). The cable 26 may include one or more of, an electrical pathway for powering the motor 20, an electrical pathway for powering one or more sensors, an electrical pathway for transmitting signals from one or more sensors associated with the blood pump 10, a fiber optic filament for transmitting signals from one or more sensors associated with the blood pump 10, or other communication conduits. In some instances, the cable 26 and associated connector 34, may be considered a single-connector cable device providing the only connection between the blood pump 10 and a medical device console controlling the blood pump 10.

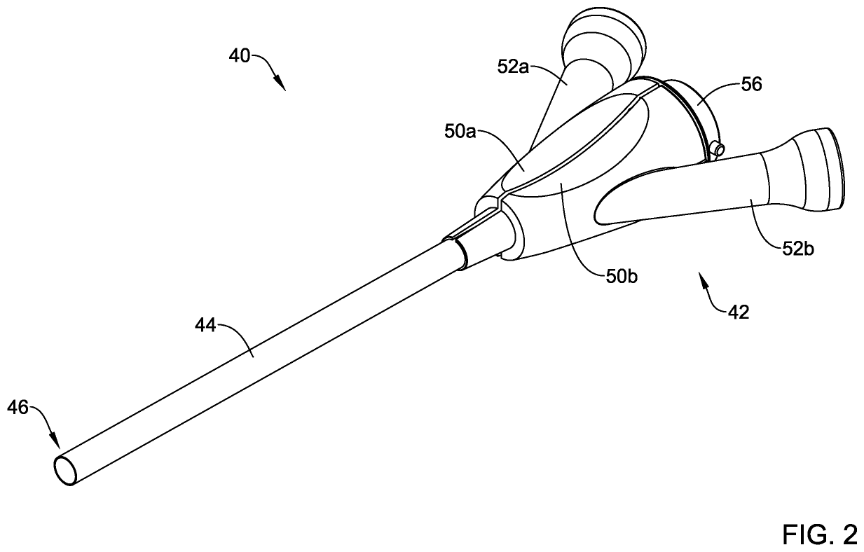

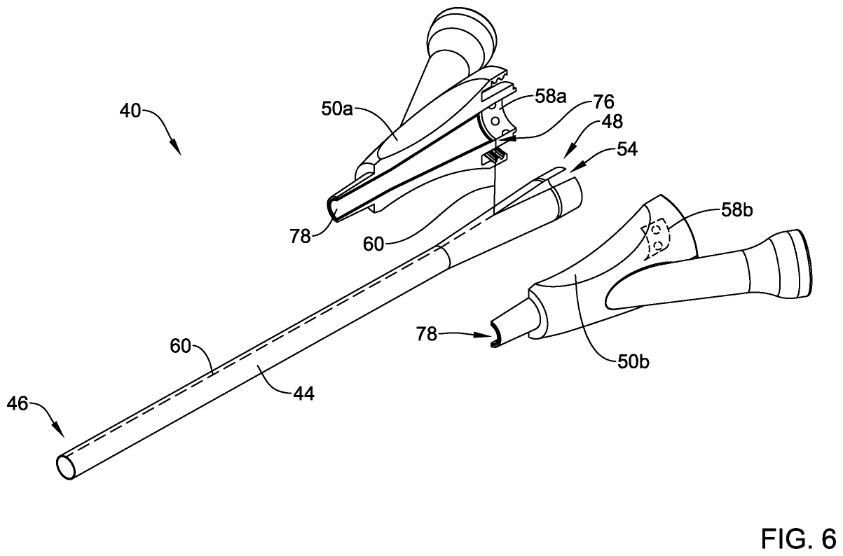

FIG. 2 is a perspective view of an example removable vascular access sheath 40 for use in providing vascular access for introducing a medical device (e.g., the percutaneous blood pump of FIG. 1) into a vasculature at a vascular access site. The access sheath 40 may include a splitable hub 42. The access sheath 40 may also include an elongate shaft 44 having a distal end region 46 and a proximal end region 48 (shown in FIG. 6) coupled to the hub 42.

The splitable hub 42 may include a first hub member 50a and a second hub member 50b releasably attached to one another. In some examples, the first hub member 50a and the second hub member 50b may be releasably attached to one another via a splitable membrane 78 (shown in FIG. 6). In some examples, the splitable membrane 78 attaching the first hub member 50a and the second hub member 50b may be positioned along an inner surface of the first hub member 50a and the second hub member 50b of the hub 42. In some examples, the splitable membrane 78 attaching the first hub member 50a and the second hub member 50b may be formed from a soft durometer material.

The hub 42 may also include a first side port 52a and a second side port 52b. In some examples, the hub may only include the first side port 52a. In some examples, the first side port 52a and a second side port 52b may be configured to permit a user to grasp each of the first side port 52a and a second side port 52b to separate the splitable hub 42. In some examples, the first side port 52a may extend at an acute angle from the first hub member 50a and the second side portion 52b may extend at an acute angle from the second hub member 50b. The first side port 52a and the second side port 52b may provide access to one or more lumens extending through the elongate shaft 44.

FIG. 2 further illustrates that the access sheath 40 may include a seal member 56. In some examples, the seal member 56 may include a hemostasis valve. The seal member 56 may be configured to seal off and minimize fluid loss during interventional and diagnostic procedures. For example, the seal member 56 may be configured to seal off and minimize fluid loss during interventional and diagnostic procedures when a guidewire or other component of another medical device is extending through the seal member 56.

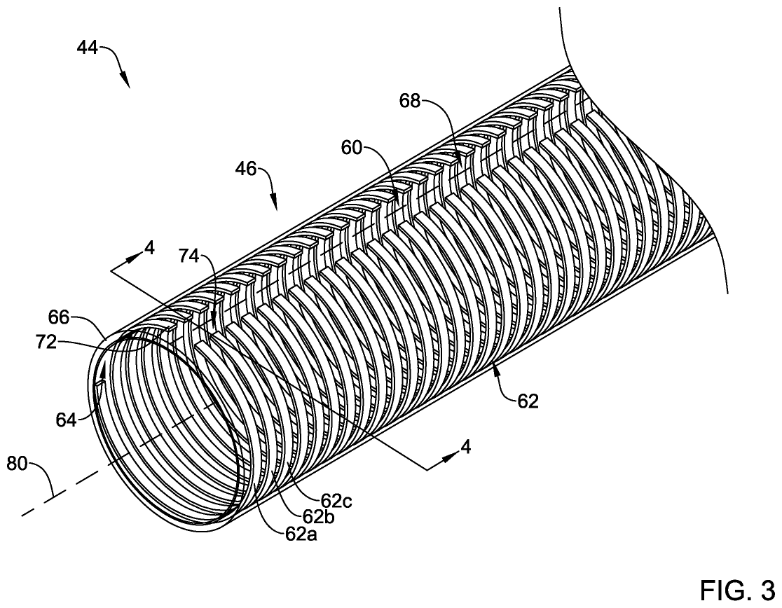

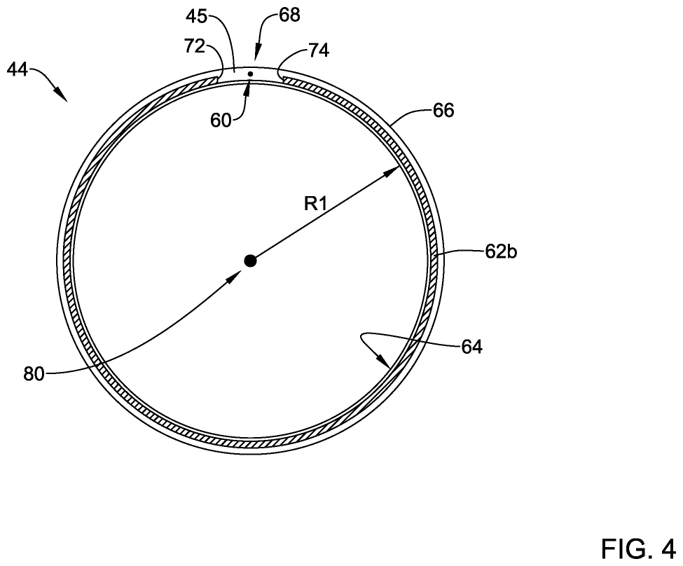

FIG. 3 illustrates the distal end region 46 of the example removeable access sheath 40 (e.g., delivery sheath, access sheath, etc.). FIG. 3 illustrates that the elongate shaft 44 of the access sheath 40 may include a plurality of reinforcing members 62 aligned along the elongate shaft 44 from the proximal end region 48 to the distal end region 46 of the elongate shaft 44. Further, it can be appreciated from FIG. 3 that each respective reinforcing member 62 may be positioned adjacent to one another along a longitudinal axis 80 of the elongate shaft 44. For example, FIG. 3 illustrates three individual reinforcing members 62a, 62b, 62c positioned adjacent to one another and aligned along the longitudinal axis 80 of the elongate shaft 44. FIG. 3 further illustrates that each of the plurality of reinforcing members 62 may partially extend about the longitudinal axis 80 of the elongate shaft 44. For example, FIG. 3 illustrates that each of the respective reinforcing members 62 may extend only partially around the longitudinal axis 80 of the elongate shaft, whereby each of the plurality of reinforcing members includes a first end circumferentially spaced away from a second end. For example, FIG. 3 illustrates that the reinforcing member 62b includes a first end 72 circumferentially spaced away from a second end 74. In some examples, the portion of the elongate shaft 44 positioned between the first ends (e.g., first end 72) and the second ends (e.g., second end 74) of each reinforcing member 62 may define a preferential separation region 68 along the elongate shaft. As will be described in greater detail below, the preferential separation region 68 may include the region along the elongate shaft 44 in which a pull cord 60 (e.g., ripcord, pull string, etc.) may pass through and slice, split, cut, etc. a wall 45 (shown in FIG. 4 and which may include the outer layer 66 and an inner liner 64) of the elongate shaft 44. FIG. 3 illustrates that, in some examples, the pull cord 60 may extend from the distal end region 46 of the elongate shaft 44 to the proximal end region 48 (shown in FIG. 6) of the elongate shaft 44. As used herein, the pull cord 60 may include a filament extending along the length of the elongate shaft 44, wherein the pull cord 60 is embedded in a portion of the wall 45 and used to facilitate separation of the wall 45 along the preferential separation region 68 when pulled through one or more polymer layers defining the wall 45.

As will be described in greater detail below, FIG. 3 illustrates that the elongate shaft 44 may further include a lubricious liner 64 extending from the distal end region 46 to the proximal end region 48. It can be appreciated from FIG. 3 that the liner 64 may be positioned radially inward of the plurality of the reinforcing members 62. It can be appreciated that, in some examples, an outer surface of the lubricious liner 64 may contact and be attached to an inner surface of the plurality of reinforcing members 62.

Additionally, the access sheath 40 may also include an outer layer 66. Outer layer 66 may extend along (e.g., cover) both the plurality of reinforcing members 62 and the liner 64. As will be discussed in greater detail below, the plurality of reinforcing members 62 may be positioned between (e.g., sandwiched between) the lubricious liner 64 and the outer layer 66. In some examples, the outer layer 66 may be attached to both the liner 64 and the plurality of reinforcing members 62. In some examples, the proximal region of the liner 64 and/or the proximal end region of the outer layer 66 may be attached to the hub 42.

FIG. 4 illustrates an example cross-sectional view of the elongate shaft 44 taken along the line 4 -4 of FIG. 3. As discussed herein, FIG. 4 illustrates the reinforcing member 62b positioned between (e.g., sandwiched between) the inner lubricious liner 64 and the outer layer 66. FIG. 4 further illustrates that the inner lubricious liner 64 may extend continuously around the longitudinal axis 80 of the elongate shaft 44. FIG. 4 further illustrates that the inner lubricious liner 64 may extend continuously around the longitudinal axis 80 of the elongate shaft 44. FIG. 4 further illustrates that the elongate shaft 44 may include a radius R₁.

Additionally, FIG. 4 illustrates that the reinforcing member 62b extends partially around the longitudinal axis 80 of the elongate shaft 44. For example, FIG. 4 illustrates that the elongate member 62b is configured such that a first end 72 of the reinforcing member 62b is spaced away from the second end 74 of the reinforcing member 62b. In some examples, the first end and the second end of each respective reinforcing member 62 may be longitudinally offset. For example, the first end 72 of the reinforcing member 62b may be longitudinally offset from the second end 74 of the reinforcing member 62b. As discussed herein, FIG. 4 also illustrates the preferential separation region 68 positioned between the first end 72 and the second end 74 of the reinforcing member 62b.

As discussed herein, the elongate sheath 44 may be configured to permit the pull cord 60 to pass through (e.g., slice, cut, split, etc.) the wall 45 of the elongate sheath along the preferential separation region 68. For example, the access sheath 40 described herein may be utilized to insert and position a relatively large medical device (e.g., blood pump 10) within a patient. However, after inserting and positioning a large medical device (e.g., blood pump 10), it may be desirable to remove the access sheath 40 from the medical device (e.g., remove it from the shaft 22 of the blood pump 10 which may be extending through the access sheath 40). Accordingly, a user may utilize the pull cord 60 to slice, split, cut, etc. the wall 45 of the elongate shaft 44 of the access sheath 40, thereby purposefully separating the wall 45 of the elongate shaft 44 along the preferential separation region 68 from the proximal end region 48 to the distal end region 46 of the elongate shaft 44 to remove the access sheath 40 from the medical device (e.g., remove it from the shaft 22 of the blood pump 10 which may be extending through the access sheath 40).

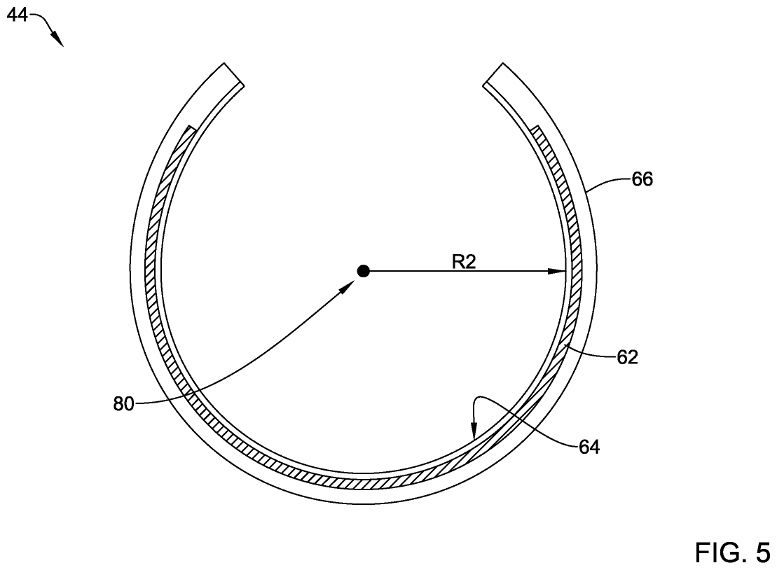

Further, in some examples, each of the reinforcing members 62 of the access sheath 40 may be configured to be positioned between the liner 64 and the outer layer 66 (such as that shown in FIG. 4) under circumferential tension. In other words, during the manufacturing process, the reinforcing members 62 as shown in FIG. 4 may be placed under circumferential tension while the liner 64 and the outer layer 66 are formed around each respective reinforcing member 62, thereby maintaining each reinforcing members 62 under circumferential tension until the pull cord 60 is utilized to separate the wall of the elongate shaft 44. It can be appreciated that the term circumferential tension, as used herein, may describe a first configuration in which the reinforcement member 62 is held under tension in a first position (as shown in FIG. 4) and whereby each reinforcement member 62 may be biased to expand radially outward to a second position (as shown in FIG. 5) when the elongate shaft 44 is split, thereby releasing each reinforcement member 62 from the first configuration and allowing each reinforcement member 62 to expand radially outward to the second position shown in FIG. 5. FIG. 5 further illustrates that the elongate shaft 44 may include a radius R₂ in the second configuration which is greater than the radius R₁ of the elongate shaft 44 shown in the first configuration of FIG. 4. In some examples, each reinforcement member 62 may be described as having an outward radial self-bias defined by the configured illustrated in FIG. 5.

FIG. 6 illustrates the first hub member 50a after being separated from the second hub member 50b. It can be appreciated that separating the first hub member 50a from the second hub member 50b may permit the hub 42 to be removed from a guidewire (or other portion of a medical device) extending through the hub 42.

FIG. 6 further illustrates the pull cord 60 having a first end 76 attached to a first lug 58a, whereby the first lug 58a may be attached to an inner surface of the first hub member 50a. Additionally, FIG. 6 illustrates that a second lug 58b may be attached to an inner surface of the second hub member 50b.

Because the pull cord 60 is attached to the first hub member 50a (via the lug 58a), FIG. 6 illustrates that separating the first hub member 50a from the second hub member 50a may initiate the pulling of the pull cord 60 from the proximal end region 48 of the elongate shaft 44. It can be further appreciated that pulling the pull cord 60 may initiate the slicing, cutting, splitting, etc. of the wall of the elongate shaft 44. Reference numeral 54 of FIG. 6 depicts the initial splitting of the wall of the elongate shaft 44.

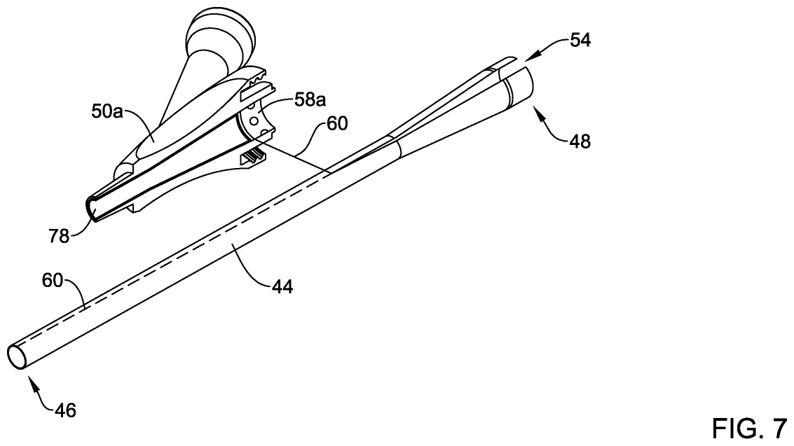

FIG. 7 illustrates the translation of the first hub member 50a in a proximal-to-distal direction to permit the pull cord 60 to cut, slice, split, etc. the wall of the elongate shaft 44 from the proximal end region 48 to the distal end region 46. It can be appreciated that a user may grasp the first hub member 50a with one hand and the elongate shaft 44 with the other hand to translate the first hub member 50a in a proximal-to-distal direction relative to the elongate member 44. Reference numeral 54 of FIG. 7 depicts the splitting of the wall of the elongate shaft 44 from the proximal end region 48 to the distal end region 46. It can be appreciated that the slicing of the wall of the elongate shaft 44 with the pull cord 60 may permit the elongate shaft 44 to be removed from a guidewire (or other portion of a medical device) extending through the lumen of the elongate shaft 44.

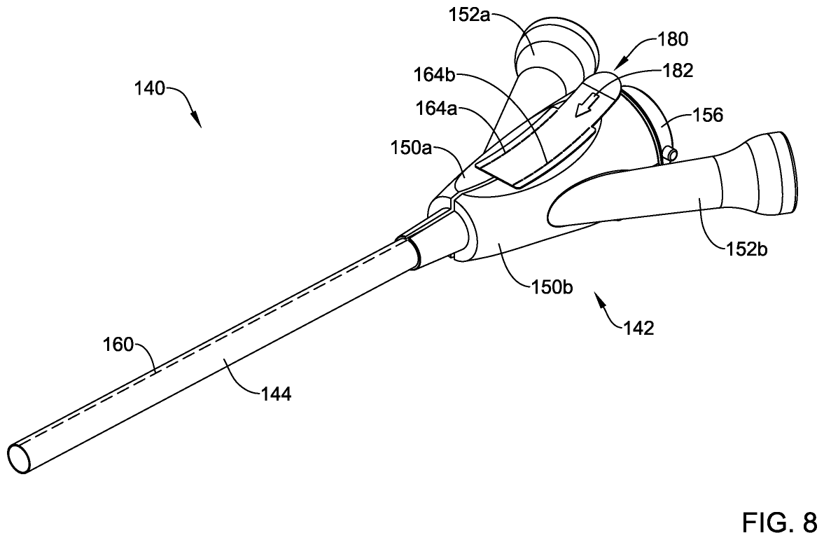

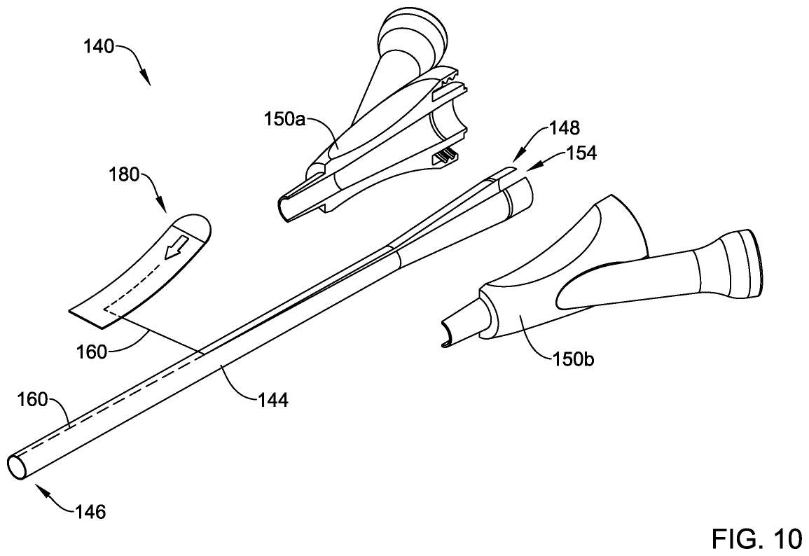

FIG. 8 is a perspective view of another example removable access sheath 140 for use in providing vascular access for introducing a medical device (e.g., the percutaneous blood pump of FIG. 1) into a vasculature at a vascular access site. The access sheath 140 may be similar in form and function to the access sheath 10. The access sheath 140 may include a splitable hub 142. The access sheath 140 may also include an elongate shaft 144 having a distal end region 146 and a proximal end region 148 (shown in FIG. 10) coupled to the hub 142.

The splitable hub 142 may include a first hub member 150a and a second hub member 150b releasably attached to one another. In some examples, the first hub member 150a and the second hub member 150b may be releasably attached to one another via a splitable membrane (similar in form and function to the splitable membrane 78 shown in FIG. 6). In some examples, the splitable membrane attaching the first hub member 150a and the second hub member 150b may be formed from a soft durometer material. The hub 142 may also include a first side port 152a and a second side port 152b. In some instances, the first side port 152a may extend at an acute angle from the first hub member 150a and the second side portion 152b may extend at an acute angle from the second hub member 150b. The first side port 152a and the second side port 152b may provide access to one or more lumens extending through the elongate shaft 144.

FIG. 8 further illustrates that the access sheath 140 may include a seal member 156. In some examples, the seal member 156 may include a hemostasis valve. The seal member 156 may be configured to seal off and minimize fluid loss during interventional and diagnostic procedures. For example, the seal member 156 may be configured to seal off and minimize fluid loss during interventional and diagnostic procedures when a guidewire or other component of another medical device is extending through the seal member 156.

FIG. 8 further illustrates that the access sheath 140 may include a pull tab 180 coupled to both the first hub member 150a and the second hub member 150b. The pull tab 180 may include a first perforation 164a and a second perforation 164b. Each of the first perforation 164a and the second perforation 164b may be generally aligned with a longitudinal axis of the elongate shaft 144. FIG. 8 illustrates that the pull tab 180 may include a symbol 182 (e.g., directional arrow) which communicates to a user the direction in which to pull the pull tab 180. FIG. 8 further illustrates that, in some examples, the access sheath may include a pull cord 160 that extends from the distal end region 146 of the elongate shaft 144 to the proximal end region 148 (shown in FIG. 10) of the elongate shaft 144. As will be discussed in greater detail below, the pull cord 160 may be attached to the pull tab 180.

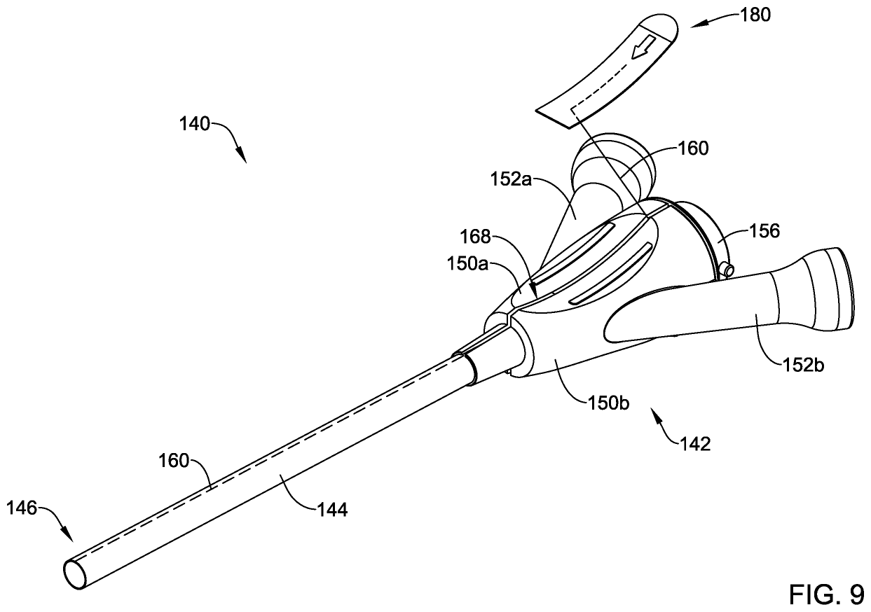

FIG. 9 illustrates the access sheath 140 whereby the pull tab 180 has been separated (e.g., pulled away from) from the first hub member 150a and the second hub member 150b. It can be appreciated from FIG. 9 that the pull tab 180 may configured to tear, rip, etc. along the first perforation 164a and a second perforation 164b when separated (e.g., pulled away from) from the first hub member 150a and the second hub member 150b.

FIG. 9 further illustrates the pull cord 160 having a first end attached to the underside of the pull tab 180. The pull cord 160 may be similar in form and function to the pull cord 60 disclosed herein. For example, the pull cord 160 may positioned within an outer layer (e.g., the outer layer 66 shown in FIGS. 3-4) of the elongate shaft 144 and extend from a proximal end region 148 (shown in FIG. 10) to a distal end region 146 of the elongate shaft 144.

FIG. 9 further illustrates that after the pull tab 180 has been separated (e.g., pulled away from) from the first hub member 150a and the second hub member 150b, the pull cord 160 may extend through a channel 168 formed between the first hub member 150a and the second hub member 150b. It can be appreciated that the channel 168 may be longitudinally aligned with the position of the pull cord 160 along the elongate shaft 144.

FIG. 10 illustrates the access sheath 140 whereby the first hub member 150a has been separated from the second hub member 150b. It can be appreciated that separating the first hub member 150a from the second hub member 150b may permit the hub 142 (together the first hub member 150a and the second hub member 150b from the hub 142 shown in FIGS. 8-9) to be removed from a guidewire (or other portion of a medical device) extending through the hub 142.

Because the pull cord 160 is attached to the pull tab 180, FIG. 10 further illustrates that separating and translating the pull tab 180 may initiate the slicing, cutting, splitting, etc. of the wall of the elongate shaft 144. For example, FIG. 10 illustrates the translation of the pull tab 180 in a proximal-to-distal direction may permit the pull cord 160 to cut, slice, split, etc. the wall of the elongate shaft 144 from the proximal end region 148 to the distal end region 146. It can be appreciated that a user may grasp the pull tab 180 with one hand and the elongate shaft 144 with the other hand to translate the pull tab 180 in a proximal-to-distal direction relative to the elongate member 144. Reference numeral 154 of FIG. 10 depicts the continued splitting of the wall of the elongate shaft 144 from the proximal end region 148 to the distal end region 146. It can be appreciated that the slicing the wall of the elongate shaft 144 with the pull cord 160 may permit the elongate shaft 144 to be removed from a guidewire (or other portion of a medical device) extending through the lumen of the elongate shaft 144.

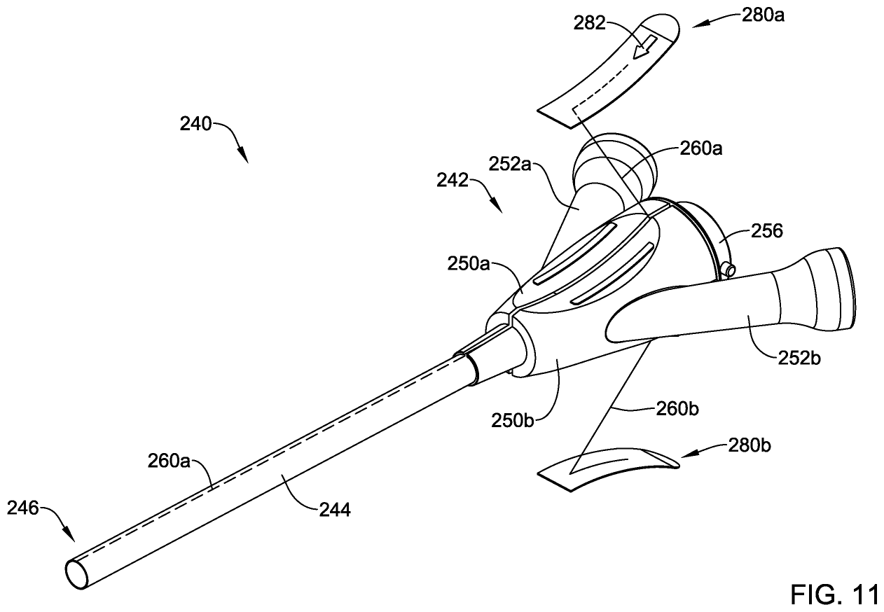

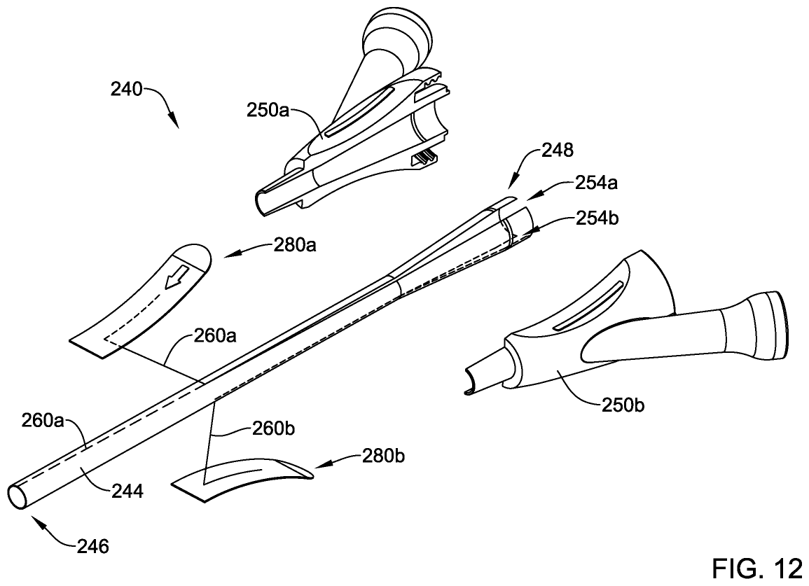

FIG. 11 illustrates another example access sheath 240. The access sheath 240 may be similar in form and function to the access sheath 140 described herein. For example, the access sheath 240 may include a splitable hub 242. The access sheath may also include an elongate shaft 244 having a distal end region 246 and a proximal end region 248 (shown in FIG. 12) coupled to the hub 242.

The splitable hub 242 may include a first hub member 250a and a second hub member 250b releasably attached to one another. In some examples, the first hub member 250a and the second hub member 250b may be releasably attached to one another via a splitable membrane (similar in form and function to the splitable membrane 78 shown in FIG. 6). In some examples, the splitable membrane attaching the first hub member 250a and the second hub member 250b may be formed from a soft durometer material. The hub 242 may also include a first side port 252a and a second side port 252b. In some instances, the first side port 252a may extend at an acute angle from the first hub member 250a and the second side port 252b may extend at an acute angle from the second hub member 250b. The first side port 252a and the second side port 252b may provide access to one or more lumens extending through the elongate shaft 244.

FIG. 11 further illustrates that the access sheath 240 may include a seal member 256. In some examples, the seal member 256 may include a hemostasis valve. The seal member 256 may be configured to seal off and minimize fluid loss during interventional and diagnostic procedures. For example, the seal member 256 may be configured to seal off and minimize fluid loss during interventional and diagnostic procedures when a guidewire or other component of another medical device is extending through the seal member 256.

FIG. 11 further illustrates that the access sheath 240 may include a first pull tab 280a coupled to both the first hub member 250a and the second hub member 250b. The first pull tab 280a may include a first perforation and a second perforation similar in form and function to the first perforation 164a and the second perforation 164b of the pull tab 180 discussed with respect to the access sheath 140. FIG. 11 illustrates that the first pull tab 280a may include a symbol 282 (e.g., directional arrow) which communicates to a user the direction in which to pull the first pull tab 280a.

FIG. 11 further illustrates that the access sheath 240 may include a second pull tab 280b coupled to both the first hub member 250a and the second hub member 250b. It can be appreciated that the second pull tab 280b may be offset from the first pull tab 280a by approximately 180 degrees. The second pull tab 280b may include a first perforation and a second perforation similar in form and function the first perforation 164a and the second perforation 164b of the pull tab 180 discussed with respect to the access sheath 140. FIG. 11 illustrates that the second pull tab 280b may include a symbol (e.g., directional arrow) similar in form and function to the symbol 282 described with respect to the first pull tab 280a.

FIG. 12 illustrates the access sheath 240 whereby the first pull tab 280a and the second pull tab 280b have been separated (e.g., pulled away from) from the first hub member 250a and the second hub member 250b. FIG. 12 further illustrates that the access sheath 240 may include a first pull cord 260a having a first end attached to the underside of the first pull tab 280a and a second pull cord 260b having a first end attached to the underside of the second pull tab 280b. The pull cords 260a, 260b may be similar in form and function to the pull cord 60 disclosed herein. For example, the first pull cord 260a may be positioned within an outer layer (e.g., the outer layer 66 shown in FIGS. 3-4) of the elongate shaft 244 and extend from a proximal end region 248 (shown in FIG. 12) to a distal end region 246 of the elongate shaft 244. Similarly, the second pull cord 260b may be positioned within an outer layer (e.g., the outer layer 66 shown in FIGS. 3-4) of the elongate shaft 244 and extend from a proximal end region 248 (shown in FIG. 12) to a distal end region 246 of the elongate shaft 244. However, it can be appreciated from FIG. 12 that the second pull cord 260b may be circumferentially offset from the first pull cord 260a approximately 180 degrees.

FIG. 12 illustrates the access sheath 240 whereby the first hub member 250a has been separated from the second hub member 250b. It can be appreciated that separating the first hub member 250a from the second hub member 250b may permit the hub 242 (together the first hub member 250a and the second hub member 250b form the hub 242 shown in FIG. 11) to be removed from a guidewire (or other portion of a medical device) extending through the hub 242.

Because the first pull cord 260a is attached to the first pull tab 280a and the second pull cord 260b is attached to the second pull tab 280b, FIG. 12 further illustrates that separating and translating both the first pull tab 280a and the second pull tab 280b may initiate the slicing, cutting, splitting, etc. of the wall of the elongate shaft 244. For example, FIG. 12 illustrates the translation of the first pull tab 280a and the second pull tab 280b in a proximal-to-distal direction may permit the first pull cord 260a and the second pull cord 260b to cut, slice, split, etc. the wall of the elongate shaft 244 from the proximal end region 248 to the distal end region 246. Reference numeral 254a of FIG. 12 depicts the continued splitting of the wall of the elongate shaft 244 from the proximal end region 248 to the distal end region 246 by the first pull cord 260a while the reference numeral 254b of FIG. 12 depicts the continued splitting of the wall of the elongate shaft 244 from the proximal end region 248 to the distal end region 246 by the second pull cord 260b. It can be appreciated that the slicing of the wall of the elongate shaft 244 with the pull cords 260a, 260b may permit the elongate shaft 244 to be removed from a guidewire (or other portion of a medical device) extending through the lumen of the elongate shaft 244.

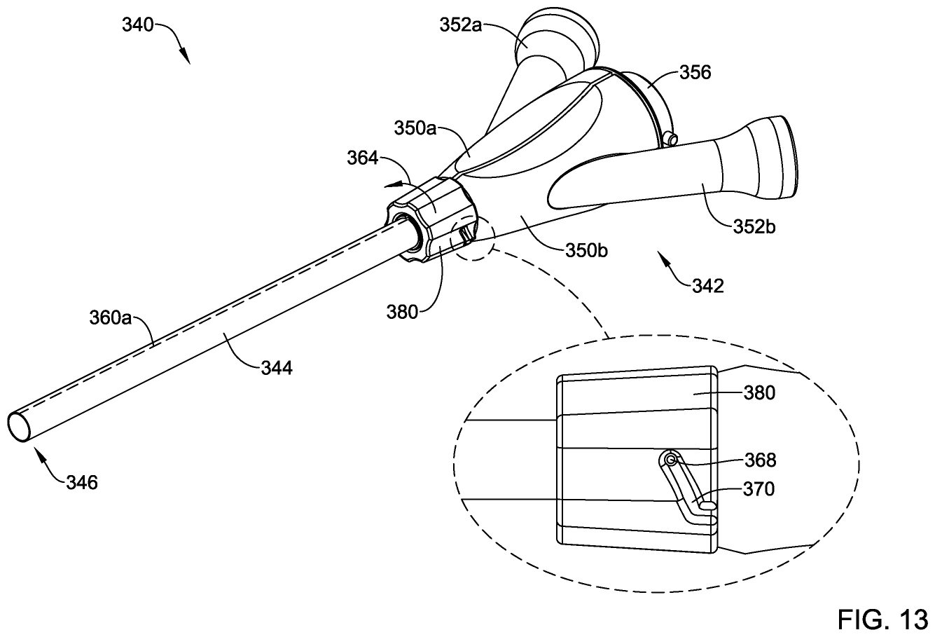

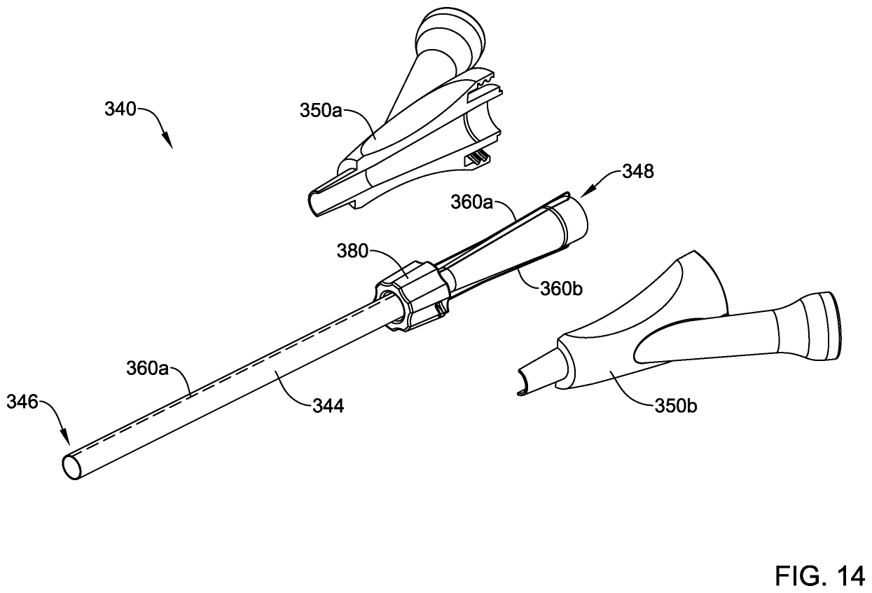

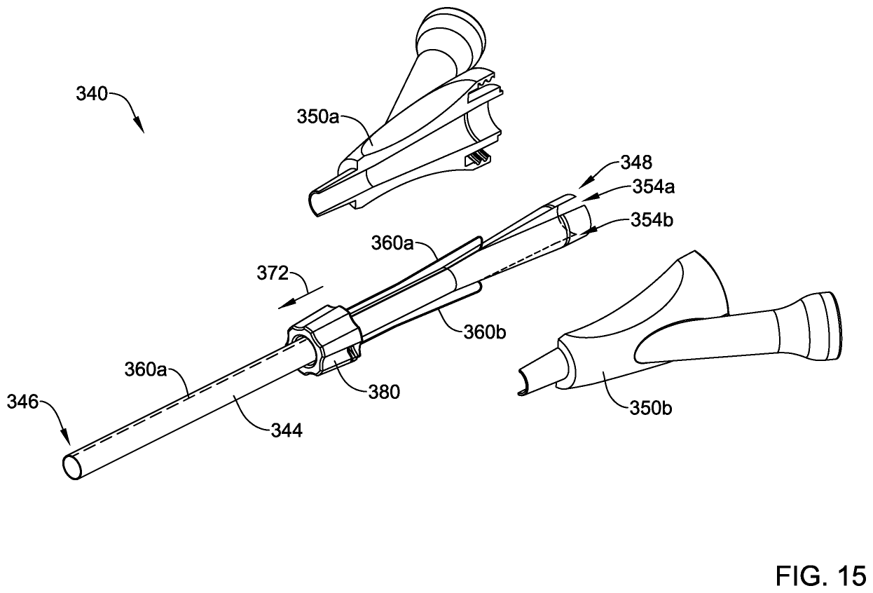

FIG. 13 is a perspective view of another example removable access sheath 340 for use in providing vascular access for introducing a medical device (e.g., the percutaneous blood pump of FIG. 1) into a vasculature at a vascular access site. The access sheath 340 may be similar in form and function to the access sheath 40. The access sheath 340 may include a splitable hub 342. The access sheath may also include an elongate shaft 344 having a distal end region 346 and a proximal end region 348 (shown in FIGS. 14-15) coupled to the hub 342.

The splitable hub 342 may include a first hub member 350a and a second hub member 350b releasably attached to one another. In some examples, the first hub member 350a and the second hub member 350b may be releasably attached to one another via a splitable membrane (similar in form and function to the splitable membrane 78 shown in FIG. 6). In some examples, the splitable membrane attaching the first hub member 350a and the second hub member 350b may be formed from a soft durometer material. The hub 342 may also include a first side port 352a and a second side port 352b. In some instances, the first side port 352a may extend at an acute angle from the first hub member 350a and the second side portion 352b may extend at an acute angle from the second hub member 350b. The first side port 352a and the second side port 352b may provide access to one or more lumens extending through the elongate shaft 344.

FIG. 13 further illustrates that the access sheath 340 may include a seal member 356. In some examples, the seal member 356 may include a hemostasis valve. The seal member 356 may be configured to seal off and minimize fluid loss during interventional and diagnostic procedures. For example, the seal member 356 may be configured to seal off and minimize fluid loss during interventional and diagnostic procedures when a guidewire or other component of another medical device is extending through the seal member 356.

FIG. 13 further illustrates that the access sheath 340 may include a pull knob 380 coupled to both the first hub member 350a and the second hub member 350b. In some examples, the pull knob 380 may include a first threaded region configured to mechanically engage threaded regions on the first hub member 350a and the second hub member 350b. In other words, the pull knob 380 may be configured to unscrew and separate from the first hub member 350a and the second hub member 350b. Rotation of the pull knob 380 is depicted by the reference numeral 364 in FIG. 13. In other examples, the pull knob 380 may be coupled to the first hub member 350a and the second hub member 350b via a friction fit configuration, whereby the pull knob 380 may be pulled off of the first hub member 350a and the second hub member 350b provided a threshold removal force is overcome.

FIG. 13 further illustrates that, in some examples, the access sheath 340 may include a first pull cord 360a that extends from the distal end region 346 of the elongate shaft 344 to the proximal end region 348 (shown in FIGS. 14-15) of the elongate shaft 344. As will be discussed in greater detail below, the first pull cord 360 may be attached to the pull knob 380.

Further, the detailed view of FIG. 13 illustrates that, in some examples, the pull knob 380 may include a channel 370 extending through the wall of the pull knob 380. Further, in some examples, the second hub member 380b may include a pin 368 configured to nest within the channel 370. It can be appreciated that the engagement of the pin 368 within the channel 370 may require a user to partially rotate the pull knob 380 in order to remove the pull knob 380 from the first hub member 350a and the second hub member 350b. It can be appreciated that the engagement of the pin 368 within the channel 370 may provide a safety feature which prevents the pull knob 380 from inadvertently being separated from the first hub member 350a and the second hub member 350b. In other words, the engagement of the pin 368 within the channel 370 may require a user to partially rotate the pull knob 380 relative to the first hub member 350a and the second hub member 350b before the pull knob 380 can be separated from the first hub member 350a and the second hub member 350b.

FIG. 14 illustrates the first hub member 350a after being separated from the second hub member 350b after the pull knob 380 has been separated from the first hub member 350a and the second hub member 350b. It can be appreciated that separating the first hub member 350a from the second hub member 350b may permit the hub 342 (together the first hub member 350a and the second hub member 350b form the hub 342 shown in FIG. 13) to be removed from a guidewire (or other portion of a medical device) extending through the hub 342.

FIG. 14 further illustrates that the access sheath 340 may include the first pull cord 360a having a first end attached to the pull knob 380 (via a first lug, for example) and a second pull cord 360b having a first end attached to the pull knob 380 (via a second lug, for example). The pull cords 360a, 360b may be similar in form and function to the pull cord 60 disclosed herein. For example, the first pull cord 360a may positioned within an outer layer (e.g., the outer layer 66 shown in FIGS. 3-4) of the elongate shaft 344 and extend from a proximal end region 348 (shown in FIGS. 14-15) to a distal end region 346 of the elongate shaft 344. Similarly, the second pull cord 360b may positioned within an outer layer (e.g., the outer layer 66 shown in FIGS. 3-4) of the elongate shaft 644 and extend from a proximal end region 348 (shown in FIGS. 14-15) to a distal end region 346 of the elongate shaft 344. However, it can be appreciated from FIG. 14 that the second pull cord 360b may be circumferentially offset from the first pull cord 360a approximately 180 degrees.

Because the first pull cord 360a is attached to the pull knob 380 and the second pull cord 360b is attached to the pull knob 380, FIG. 15 illustrates that translating the pull knob 380 may initiate the slicing, cutting, splitting, etc. of the wall of the elongate shaft 344. For example, FIG. 15 illustrates the translation of the pull knob 380 in a proximal-to-distal direction may permit the first pull cord 360a and the second pull cord 360b to cut, slice, split, etc. the wall of the elongate shaft 344 from the proximal end region 348 to the distal end region 346. Translation of the pull knob 380 in a proximal-to-distal direction is depicted by the reference numeral 372 in FIG. 15. Reference numeral 354a of FIG. 15 depicts the continued splitting of the wall of the elongate shaft 344 from the proximal end region 348 to the distal end region 346 by the first pull cord 360a while the reference numeral 354b of FIG. 15 depicts the continued splitting of the wall of the elongate shaft 344 from the proximal end region 348 to the distal end region 346 by the second pull cord 360b. It can be appreciated that the slicing the wall of the elongate shaft 344 with the pull cords 360a, 360b may permit the elongate shaft 344 to be removed from a guidewire (or other portion of a medical device) extending through the lumen of the elongate shaft 344.

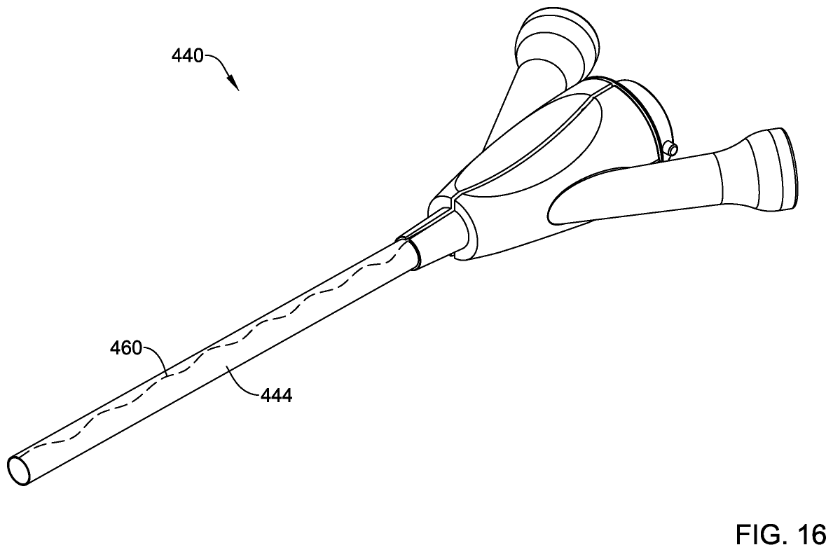

While the example pull cords described herein (e.g., pull cord 60) are depicted as being aligned in with the longitudinal axis of the elongate shaft in which they are positioned, in some examples the pull cords described herein may include one or more curves and/or a wavy configuration within the elongate shaft. It can be appreciated that a curved or wavy configuration of the pull cord may provide additional radial strength to the elongate shaft in which a respective pull cord is positioned. For example, FIG. 16 illustrates an example access sheath 440 having a pull cord 460 including a curved configuration within an example elongate shaft 444.

In some examples, access sheath 40 (or other access sheaths and components thereof described herein) may be made from materials such as metals, metal alloys, polymers, ceramics, metal-polymer composites, or other suitable materials, and the like. Some examples of suitable materials may include metallic materials such as stainless steels (e.g. 304v stainless steel or 316L stainless steel), nickel-titanium alloys (e.g., nitinol, such as super elastic or linear elastic nitinol), nickel-chromium alloys, nickel-chromium-iron alloys, cobalt alloys, nickel, titanium, platinum, or alternatively, a polymeric material, such as a high performance polymer, or other suitable materials, and the like. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

In some examples, the access sheath 40 (or other access sheaths and components thereof described herein, including, but not limited to the pull tabs, knobs, pull cords, etc. disclosed herein) may be made from materials such as, for example, a polymeric material, a ceramic, a metal, a metal alloy, a metal-polymer composite, or the like. Examples of suitable polymers may include nylons, acrylonitrile butadiene styrene (ABS), polyurethane, a polyether-ester such as ARNITEL® available from DSM Engineering Plastics, a polyester such as HYTREL® available from DuPont, a linear low density polyethylene such as REXELL®, a polyamide such as DURETHAN® available from Bayer or CRISTAMID® available from Elf Atochem, an elastomeric polyamide, a block polyamide/ether, a polyether block amide such as PEBA available under the trade name PEBAX®, silicones, polyethylene, Marlex high-density polyethylene, polyetheretherketone (PEEK), polyimide (PI), and polyetherimide (PEI), a liquid crystal polymer (LCP) alone or blended with other materials. In some examples, a suitable polymeric material may have a yield strain of at least 20%, at least 30%, at least 40%, at least 50%, or more. In some examples, the sheath, the membrane, the liner or other components of the access sheaths disclosed hereinmay be made from a material having a low coefficient of friction. In some examples, the sheath, the membrane, the liner or other components of the access sheaths disclosed herein may be formed from a fluoropolymer, such as polytetrafluoroethylene (PTFE) or fluorinated ethylene propylene (FEP).

Portions of access sheath 40 (or other access sheaths and components thereof described herein) may be made of, may be doped with, may include a layer of, or otherwise may include a radiopaque material. Radiopaque materials are understood to be materials capable of producing a relatively bright image on a fluoroscopy screen or another imaging technique such as X-ray during a medical procedure. This relatively bright image aids the user of device in determining its location. For example, one or more of the elements described above (i.e., the sheath, the membrane, the medical device, etc.) may include or be formed from a radiopaque material. Suitable materials can include, but are not limited to, bismuth subcarbonate, iodine, gold, platinum, palladium, tantalum, tungsten or tungsten alloy, and the like.

It should be understood that although the above discussion was focused on percutaneous medical procedures within the vasculature of a patient, other examples or methods in accordance with the disclosure can be adapted and configured for use in other parts of the anatomy of a patient. For example, devices and methods in accordance with the disclosure can be adapted for use in the digestive or gastrointestinal tract, such as in the mouth, throat, small and large intestine, colon, rectum, and the like. For another example, devices and methods can be adapted and configured for use within the respiratory tract, such as in the mouth, nose, throat, bronchial passages, nasal passages, lungs, and the like. Similarly, the devices and methods described herein with respect to percutaneous deployment may be used in other types of surgical procedures as appropriate. For example, in some examples, the devices may be deployed in a non-percutaneous procedure. Devices and methods in accordance with the disclosure can also be adapted and configured for other uses within the anatomy.

It should be understood that this disclosure is, in many respects, only illustrative. Changes may be made in details, particularly in matters of shape, size, and arrangement of steps without exceeding the scope of the disclosure. The invention's scope is, of course, defined in the language in which the appended claims are expressed.

What is claimed is:

1. An access sheath for a medical device, comprising:
an elongate shaft having a distal end region, a proximal end region and a wall;
a hub coupled to the proximal end region of the elongate shaft; and
a first pull cord coupled to the hub, wherein the first pull cord is configured to translate along the elongate shaft from the proximal end region to the distal end region;
wherein translation of the first pull cord along the elongate shaft cuts the wall of the elongate shaft from the proximal end region to the distal end region.

2. The access sheath of claim 1, wherein the hub is configured to separate into a first hub member and a second hub member.

3. The access sheath of claim 2, wherein the first pull cord includes a first end attached to the first hub member.

4. The access sheath of claim 1, wherein at least a portion of the first pull cord is positioned within the wall of the elongate shaft.

5. The access sheath of claim 1 further comprising a second pull cord coupled to the hub.

6. The access sheath of claim 5, wherein the second pull cord is circumferentially offset from the first pull cord.

7. The access sheath of claim 6, wherein at least a portion of the second pull cord is positioned within the wall of the elongate shaft.

8. The access sheath of claim 7, wherein the second pull cord is configured to translate along the elongate shaft from the proximal end region to the distal end region, and wherein translation of the second pull cord along the elongate shaft cuts the wall of the elongate shaft from the proximal end region to the distal end region.

9. The access sheath of claim 1, further comprising a first pull tab releasably coupled to the hub.

10. The access sheath of claim 9, wherein a first end of the first pull cord is attached to the first pull tab.

11. The access sheath of claim 10, further comprising a second pull tab releasably coupled to the hub and a second pull cord attached to the second pull tab.

12. The access sheath of claim 1, further comprising a pull knob releasably attached to the hub.

13. The access sheath of claim 12, wherein a first end of the first pull cord is attached to the pull knob.

14. The access sheath of claim 13, further comprising a second pull cord attached to the pull knob.

15. The access sheath of claim 2, wherein the first hub member is coupled to the second hub member via a splitable membrane.

16. The access sheath of claim 15, wherein at least a portion of the splitable membrane is positioned along an inner surface of the hub.

17. An access sheath for a medical device, comprising:
an elongate shaft having a distal end region, a proximal end region and a wall;
a hub coupled to the proximal end region of the elongate shaft, wherein the hub includes a first hub member configured to separate from a second hub member;
a pull cord coupled to the hub, wherein the pull cord is configured to translate along the elongate shaft from the proximal end region to the distal end region;
wherein translation of the pull cord along the elongate shaft cuts the wall of the elongate shaft from the proximal end region to the distal end region.

18. The access sheath of claim 17, wherein the hub is configured to separate into a first hub member and a second hub member.

19. The access sheath of claim 18, wherein the pull cord includes a first end attached to the first hub member.

20. A method for advancing a medical device into a body lumen, comprising:
positioning an access sheath adjacent a body lumen, the access sheath comprising:
an elongate shaft having a distal end region, a proximal end region and a wall;
a hub coupled to the proximal end region of the elongate shaft; and
a pull cord attached to the hub, the pull cord configured to cut the wall of the elongate shaft;
advancing the medical device through the access sheath and into the body lumen;
translating the pull cord along the elongate shaft from the proximal end region to the distal end region, wherein translation of the pull cord along the elongate shaft cuts the wall of the elongate shaft from the proximal end region to the distal end region; and
removing the access sheath from the medical device via passing at least a portion of the medical device through the cut portion of the elongate shaft.
